(19) United States
(12) Patent Application Publication
(10) Pub. No.: US 2007/0014740 A1
(43) Pub. Date: Jan. 18, 2007

(54) Oral compositions having cationic active ingredients

(75) Inventors: Jeffrey Miller, Sayreville, NJ (US); Thomas J. Boyd, Metuchen, NJ (US); Evangelia S. Arvanitidou, Princeton, NJ (US); Michael Prencipe, West Windsor, NJ (US); Andre' Morgan, Robbinsville, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 11/182,398

(22) Filed: Jul. 15, 2005

(51) Int. Cl. A61K 8/46

(52) U.S. Cl. 424/49; 424/57

(57) ABSTRACT

An oral composition is provided covering one or more cationic active ingredients and a cationic-compatible inorganic particulate having a porous surface that is substantially inert to the cationic active ingredient. The oral composition also has a cationic-compatible surfactant system and a carrier that is stable in the presence of the cationic active ingredient. The oral composition can be rpvioded in a dual tube or dingle tube form, if desired.

### BACKGROUND OF THE INVENTION

Dental plaque or plaque bio-film is a soft deposit that forms on teeth and is implicated in the occurrence of gingivitis and other forms of periodontal disease. Various cationic antibacterial agents have found to have the clinical ability to retard the growth of bacterial and hence have the ability to minimize plaque formation, oral infections and dental disease associated therewith. Many cationic active ingredients are theorized to have antimicrobial action due to their ability to bind to negatively-charged protein moieties on bacterial cells present in the mouth. For example, cetyl pyridinium chloride (CPC) is believed to function in this manner and is regarded as an effective antibacterial/antiplaque active ingredient for oral compositions. Other cationic actives are theorized to function as anti-attachment compounds that prevent attachment of bacteria to enamel tooth surfaces by modifying the surface energy of the enamel. Such actives include antibacterial amino acid derivative esters, such as ethyl lauroyl arginine. Accordingly, the efficacy of these cationic active ingredients' function is dependent upon preserving their cationic properties in vivo to prevent the formation of plaque, gingivitis, and cavities.

However, there are difficulties associated with providing a stable oral care composition that preserves the cationic nature of the cationic active ingredients, and their bioavailability and efficacy. This is particularly difficult because many conventional oral care ingredients, such as inorganic particulate abrasives and surfactants, have an anionic (negative) nature. Cationic active ingredients are potentially attracted and bound to such negatively-charged ingredients, and hence are prevented from performing their intended function. Although the cationic active material ingredients, such as for example, antibacterial amino acid ester compounds, are effective antibacterial agents in vitro, these ingredients have often been observed to not exhibit the desired efficacy when applied in vivo by oral composition.

### BRIEF SUMMARY OF THE INVENTION

In various embodiments, the present invention provides oral compositions that comprise a cationic active ingredient and a cationic-compatible inorganic particulate having a surface that is substantially inert to the cationic active ingredient. The oral composition also preferably comprises a cationic-compatible surfactant system.

In one aspect, the present invention provides an oral composition comprising:

(a) a compound represented by formula (I) 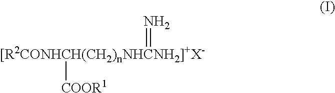
where R¹ and R² are alkyl groups, and X is an anion. In preferred embodiments, R¹ is an alkyl chain having between 1 to 8 carbon atoms and R² is alkyl chain of 6 to 30 carbon atoms. The oral composition also comprises a cationic-compatible inorganic particulate having a surface that is substantially inert to the cationic antibacterial ester. The cationic-compatible surfactant system preferably comprises a surfactant selected from the group consisting of: polyoxyethylene sorbitan monolaurate, cocoamido propyl betaine, poly(oxyethylene)-poly(oxypropylene)(poloxamer), and sodium methyl cocoyl taurate.

### DETAILED DESCRIPTION OF THE INVENTION

The present invention provides compositions comprising one or more cationic active ingredients. Such cationic ingredients include any material comprising a cationic (positively-charged) moiety. Cationic active ingredients are, for example, those which if in an aqueous composition, chemically react with an anionic dentifrice component (e.g., conventional abrasives, anionic active ingredients, or anionic surfactants), such that the efficacy of the ingredient is substantially reduced.

Cationic active ingredients among those useful herein include materials operable to treat or prevent a disorder or provide a cosmetic benefit. In various embodiments, the active is a “systemic active” which is operable to treat or prevent a disorder which, in whole or in part, is not a disorder of the oral cavity. In various embodiments, the active is an “oral care active” operable to treat or prevent a disorder or provide a cosmetic benefit within the oral cavity (e.g., to the teeth, gingival or other hard or soft tissue of the oral cavity).

Oral care actives among those useful herein include antibacterial agents, anti-inflammatory agents, anticaries agents, tartar control agents, antiplaque agents, periodontal actives, breath freshening agents, malodor control agents, tooth desensitizers, salivary stimulants, and combinations thereof. It is understood that while general attributes of each of the above categories of actives may differ, there may some common attributes and any given material may serve multiple purposes within two or more of such categories of actives.

In a preferred embodiment, at least one of the active ingredients has an antibacterial and/or antiplaque oral care benefit. In one embodiment, the cationic active material has an anti-attachment mechanism.

Suitable cationic antibacterial agents for use in oral compositions of the invention include, for example:

(i) quaternary ammonium compounds, such as those in which one or two of the substituents on the quaternary nitrogen has from 8 to 20, preferably from 10 to 18 carbon atoms and is preferably an alkyl group, which may optionally be interrupted by an amide, ester, oxygen, sulfur, or heterocyclic ring, while the remaining substituents have a lower number of carbon atoms, for instance from 1 to 7, and are preferably alkyl, for instance methyl or ethyl, or benzyl. Examples of such compounds include benzalkonium chloride, dodecyl trimethyl ammonium chloride, benzyl dimethyl stearyl ammonium chloride, hexadecyltrimethyl ammonium bromide, benzethonium chloride (diisobutyl phenoxyethoxyethyl dimethyl benzyl ammonium chloride) and methyl benzethonium chloride;

(ii) pyridinium and isoquinolinium compounds, including hexadecylpyridinium chloride and alkyl isoquinolinium bromides;

(iii) pyrimidine derivatives such as hexetidine (5-amino-1,3-bis(2-ethylhexyl)-5-methyl-hexahydropyrimidine);

(iv) amidine derivatives such as hexamidine isethionate (4,4′-diamidino-αω-diphenoxy-hexane isethionate);

(v) bispyridine derivatives such as octenidine dihydrochloride (N,N′[1,10-decanediyldi-1(4H)-pyridinyl-4-ylidene]-bis(1-octanamine)dihydrochloride);

(vi) guanides, for example, mono-biguanides such as p-chlorobenzyl-biguanide and N′(4-chlorobenzyl)-N″-(2,4-dichlorobenzyl)biguanide, poly(biguanides) such as polyhexamethylene biguanide hydrochloride, and bis-biguanides of the general formula (1): 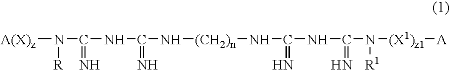
in which A and A¹ each represent (i) a phenyl group optionally substituted by (C₁₋₄)alkyl, (C₁₋₄)alkoxy, nitro, or halogen, (ii) a (C₁₋₁₂)alkyl group, or (iii) a (C₄₋₁₂)alicyclic group; X and X¹ each represent (C₁₋₃)alkylene; R and R¹ each represent hydrogen, (C₁₋₁₂)alkyl, or aryl (C₁₋₆)alkyl; Z and Z1 are each 0 or 1; n is an integer from 2 to 12; and the polymethylene chain (CH₂)ₙ may optionally be interrupted by oxygen or sulfur or an aromatic (for instance phenyl or naphthyl) nucleus; and orally acceptable acid addition salts thereof; examples of such bis-biguanides include chlorhexidine and alexidine. Suitable acid addition salts of the bis-biguanides of general formula (1) include the diacetate, the dihydrochloride and the digluconate. Suitable acid addition salts of chlorhexidine are those which have a water solubility at 20° C. of at least 0.005% w/v and include the digluconate, diformate, diacetate, dipropionate, dihydrochloride, dihydroiodide, dilactate, dinitrate, sulphate, and tartrate salts. Preferably the salt is the dihydrochloride, diacetate or digluconate salt of chlorhexidine. Suitable acid addition salts of alexidine include the dihydrofluoride and the dihydrochloride salts; and

(vii) N^{α}-acyl amino acid alkyl esters and salts, more particularly, N^{α}-acyl arginine alkyl esters and salts generally represented by the formula (2) below: 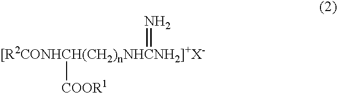
where both R¹ and R² are alkyl groups. In particular, R¹ is preferably an alkyl chain of 1 to 8 carbon atoms, preferably from 1 to 3 carbon atoms, and most preferably 3 carbon atoms; R² is an alkyl chain of 6 to 30 carbon atoms, preferably from 10 to 12 carbon atoms, and mixtures thereof; and X is an anion. In various embodiments, the R²CO moiety comprises a natural fatty acid residue such as a natural fatty acid selected from the group consisting of coconut oil fatty acid, tallow fatty acid residue, or a mono-fatty acid residue such as selected from the group consisting of lauroyl(C₁₂), myristyl(C₁₄), stearoyl (C₁₈) fatty acid residues, and mixtures thereof. In one embodiment, the R²CO moiety comprises a lauroyl fatty acid residue. X may be any counter-anion that provides a reasonable degree of solubility in water (preferably at least about 1 g in 1 L of water). Examples of X counter anions which form antibacterial ester salts of the above identified formula, include inorganic acid salts, such as those comprising halogen atoms (e.g., chloride or bromide) or dihydrogen phosphate, or an organic salt such as acetate, tautarate, citrate, or pyrrolidone-carboxylate (PCA). The chloride salt is preferred.

Examples of antibacterial ester compounds preferred in the practice of the present invention include antibacterial ester compounds of the above-identified formula wherein n in the formula equals 3. Such compounds include N^{α}-acyl arginine alkyl esters and salts thereof, such as N^{α}-cocoyl-L-arginine methyl ester, N^{α}-cocoyl-L-arginine ethyl ester, N^{α}-cocoyl-L-arginine propyl ester, N^{α}-stearoyl-L-arginine methyl ester, N^{α}-stearoyl-L-arginine ethyl ester hydrochloride. In one embodiment, the arginine derivative compound is the hydrogen chloride salt of ethyl lauroyl arginine (ELAH).

In certain embodiments of the present invention, the cationic active ingredient included in the oral composition is selected from one or more of: benzethonium chloride, octenidine, hexetidine, hexamidine, cetyl pyridinium chloride, chlorhexidine, alexidine, and N^{α}-acyl arginine alkyl ester salts. In one embodiment, the cationic active ingredient comprises cetyl pyridinium chloride (CPC). In another embodiment, the cationic active ingredient comprises an ethyl lauroyl arginine ester hydrochloride (ELAH). In yet other embodiments, the cationic active ingredient may include multiple active compounds. For example, in one embodiment, the cationic active ingredient comprises both cetyl pyridinium chloride (CPC) and ethyl lauroyl arginine ester hydrochloride (ELAH). As recognized by one of skill in the art, other combinations of cationic active ingredients are contemplated for the present invention. Likewise, as will be described in greater detail below, the oral composition may further comprise additional active ingredients that do not adversely affect the cationic active ingredients described above.

Advantageously, the cationic antibacterial active ingredient is present in the range of about 0.005 to about 10% by weight. In various embodiments, the cationic antibacterial active ingredient is present in the oral composition from about 0.005 to about 5%, more preferably from about 0.05 to about 3%.

In various embodiments of the present invention, a cationic-compatible inorganic particulate is used in the oral care composition with the cationic active ingredient. Without limiting the compositions, methods or utility of the present invention, in various embodiments the inorganic particulate components are believed to afford diminished interaction between cationic active compounds and the inorganic particulates, thus increasing the bioavailability of the cationic active compounds.

In various embodiments of the present invention, the oral composition comprises a cationic-compatible inorganic particulate having a surface that is substantially inert to the cationic active ingredient. “Substantially inert” as used herein refers to the surface of the cationic-compatible inorganic particulate having minimal undesirable interaction with the cationic active ingredients. A substantially inert surface can be quantified by a variety of methods. The inertness of the surface may be achieved in any way, including having a porous surface, a coated surface, and/or a textured surface.

For example, the improved compatibility of cationic-compatible inorganic particulates having substantially inert porous surfaces of the present invention over conventional inorganic particulates can be shown for example, from in vitro measurements of availability of the various antimicrobial cationic agents of the compositions of the invention. One such example is by combining a cationic active with the particulate in an aqueous solution and determining the amount of cationic active recovered, which can be expressed as the % of recovery of the active ingredient from the solution, as “% recovery value.” Since the % recovery indicates how much of the cationic active was not undesirably bound to the particulate, this method provides an empirical method of evaluating cationic compatibility.

In various embodiments, a substantially inert surface of a porous inorganic particulate for use with the present invention preferably has a % recovery value of at least about 50%, particularly greater than 60%, particularly greater than 70%, more particularly greater than 80%, and even more particularly greater than 90% with the cationic active ingredients. Such a testing procedure can be conducted with a cationic active, such as CPC. For example, in one procedure, a 0.3% solution of CPC is provided at 27 grams and mixed with 3 grams of the inorganic particulate to be tested (e.g., silica or dicalcium phosphate). The mixture is shaken for 10 minutes and then placed in a 60° C. aging oven for 7 days. After 7 days, the sample is centrifuged and micro-filtered, and then diluted by 20 fold. Analysis of the available CPC in the supernatant is conducted by measuring absorbance (for example, at 268 nm) and relating this data to the amount of CPC recovered from the original amount placed in the mixer. The % recovery value is the difference between the original amount of CPC placed in the mixture to the amount of CPC recovered, divided by the original amount of CPC.

While not wishing to be limited by any particular theory, it is believed that in certain embodiments, a porous surface of an inorganic particulate that has a surface charge that is cationic, neutral, or only slightly anionic is substantially inert to cationic active ingredients. In certain embodiments, the inorganic particulate has a low surface area and oil of absorption, including surface-modified silica products which have diminished interaction with cationic active ingredients. It has been theorized that such surface modification of the silica particulates diminishes the adsorbent properties of the porous surface and/or diminishes the potential for the cationic active ingredients to approach and interact with the charged surface(s).

By way of background, the surface charge density (σ) can be expressed as:

$$σ = \frac{q}{A} \qquad ( 3 )$$

that reflects the total amount of electrical charge (q) per unit area (A). Often surface charge is expressed for particulates as the point of zero charge (pH^{PZC}), which coincides with the pH conditions where the surface charge σ equals 0 C/m², reflecting a balance of negative and positive charges on the surface of the particulate. Other factors that impact the surface charge include for example, the crystal structure, any edges, kinks, or structural features, surface area, pore size, and the like.

Typical silica based particulates for use in oral care compositions are precipitated amorphous silica (SiO₂), which can be formed by combining sodium silicate (Na₂OₓSiO₂) with a strong acid, such as sulfuric acid (H₂SO₄). The surface of precipitated silica particulates formed in this reaction can comprise hydroxyl groups (Si—O—H), and potentially silanediol (—Si—(OH)₂), silanetriol (—Si—(OH)₃), surface bound water, and the like. Such hydroxyl species tend to lend anionic or negative charge to the surface of a particle. One example of determining the surface charge of a silica particle is provided in U.S. Pat. No. 5,616,316 to Persello, the contents of which are incorporated herein by reference.

Preferably, such a particle has a mean particle size of up to 20 microns in diameter. In various embodiments, it is preferred that the cationic-compatible inorganic particulate has a porous surface that has a BET (Brunauer Emmett and Teller method) surface area of less than about 100 m²/g. In other embodiments, the cationic-compatible inorganic particulate has a DBP (dibutyl phthalate) oil of absorption reflecting the structure (or morphology) of the particles of less than about 150 g/100 g. In other embodiments, the cationic-compatible inorganic particulate has a BET surface area of less than about 100 m²/g and further has a DBP structure of less than about 150 ml/100 g.

In one embodiment, the oral composition comprises an inorganic particulate that has a low surface charge, preferably with a pH_{PZC} of 5 to 7, such as with calcium phosphate particulates, including dicalcium phosphate, more particularly dicalcium phosphate dihydrate (CaHPO₄.2H₂O). The dicalcium phosphate particulate has a typical surface area of less than about 1.7 m²/g and an DPB oil of absorption of less than about 50 ml/100 g.

In other embodiments, the cationic-compatible porous inorganic phosphate is a low surface area, or surface-modified, silica particulate. In certain embodiments, the surface-modified precipitated silica product comprises surface-treated silica particulates having a median diameter of 1 to 100 micrometers. For purposes herein, the term “silica particulate” means finely divided silica, and the term encompasses silica primary particles, silica aggregates (i.e., unitary clusters of a plurality of silica primary particles), silica agglomerates (i.e., unitary clusters of a plurality of silica aggregates), singly or in combinations thereof.

These silica particulates support deposits of a relatively denser amorphous “active” silica material. The silica material is deposited in an amount effective to provide a BET specific surface area of from 1 to 50 square m²/g, preferably 1 to 40 m²/g, more preferably less than 30 m²/g that are amounts effective to render the silica surface as being substantially inert to the cationic active ingredients. In one particular aspect, the silica particulate is in the form of silica aggregates or agglomerates formed of the silica particles. Thus, in the certain embodiments, the low surface area modified abrasive has a median diameter of less than about 100 micrometers and a BET surface area of less than about 50 m²/g.

The modified low-surface area silica product may be produced via a process where silica particulate is a preformed material or formed in-situ, followed by precipitating active silica upon the silica particulate effective to satisfy the specific surface area and reduced cationic active attachment requirements described elsewhere herein. The denser silica material deposited on the silica particulate “coats” the underlying silica substrate particulate primarily in the sense that it penetrates into and/or blocks the opening of the pores of the underlying silica particulate to effectively reduce the surface area of the silica particulate substrate. Quantitative BET surface area measurements taken before and after deposition of the active silica on the silica substrate particulate can be compared to determine qualitatively if a less porous (i.e., more dense) particulate has been created, as indicated by a measurable reduction in the specific surface area value. Such a modified low-surface area silica particulate that is cationic-compatible is disclosed, for example, in U.S. Patent Application Publication 2004/0161389 to Gallis et al., which is incorporated herein by reference.

Another suitable modified low-surface area silica that is suitable for use in the present invention as a cationic-compatible inorganic particulate that is substantially inert to the cationic active ingredient is described in U.S. Patent Application Publication 2004/0616390 to Gallis et al., which is incorporated herein by reference. In one embodiment, the modified low-surface area silica products can be produced by providing porous silica substrate particles in the same manner as described above. A dense silica material is deposited onto the silica substrate particles effective to penetrate into and/or block at least part of the pore openings on the silica substrate particles to reduce the pores having a size greater than about 500 A, which in turn limits the cumulative surface area for those large diameter pores on the surface-treated silicas to less than approximately 8 m²/g, as measured by mercury intrusion porosimetry. Pores sizes of greater than about 500 A, i.e., large diameter pores, appear to be more accessible to certain cationic active compounds, such as CPC, as compared to pores having smaller sizes. Thus, reducing the pores on the silica particles having sizes of greater than about 500 A appears to effectively limit the interaction of the cationic active ingredient with the pores at the surfaces of the silica particles.

In various embodiments, the modified low-surface area silica particulates preferably have a % CPC recovery value of at least 50%, particularly greater than 60%, and more particularly greater than 70%, and even more particularly greater than 80%, and it generally ranges between about 55% to about 95%. These % CPC recovery values are attainable due to the treatment of the silica substrate particles effective to reduce the surface pores having a size greater than about 500 A such that the cumulative surface area of those sized pores is preferably less than about 8 m²/g, and preferably less than about 7 m²/g, and more preferably less than about 6 m²/g, as measured by mercury intrusion porosimetry.

Such a silica product in the present embodiment can be produced by a general process scheme, in which: 1) a slurry of amorphous silica particles is provided either by slurrying up a prefabricated silica material obtained in dry finely divided form, or, alternatively, from an ongoing production run in which fresh precipitated silica is in slurry or wet cake form without ever having been dried into powder form, followed by: 2) precipitating active silica upon the substrate silica particles effective to reduce the cumulative surface area of all pores having sizes greater than about 500 A to less than about 8 m²/g. For purposes of measuring BET surface area, N₂ physisorption is commonly used. However, because of the size of nitrogen gas, there are pores contributing to the overall surface area on silica particles that are accessible to the gaseous N₂ used in conventional BET measurements, but which are not readily accessible to larger cationic active, such as CPC. Surface area resulting from micropores may be accessible to gaseous nitrogen (as measured by N₂ physisorption), but is not readily accessible to an aqueous slurry of CPC. Consequently, it is not possible to use BET N₂ surface area measurements per se to identify silica particles having the favorable pore size distributions described herein for obtaining % CPC recovery values of greater than approximately 55%. Instead, mercury intrusion porosimetery is preferably used in embodiments of the present invention as the method for measuring cumulative surface area of the silica particles at the identified critical pore size values.

As is generally known, the mercury porosimetry technique is based on the intrusion of mercury into a porous structure under stringently controlled pressures. From the pressure versus intrusion data, the instrument generates volume and size distributions using the Washburn equation. Since mercury does not wet most substances and will not spontaneously penetrate pores by capillary action, it must be forced into the pores by the application of external pressure. The required pressure is inversely proportional to the size of the pores, and only slight pressure is required to intrude mercury into large macropores whereas much greater pressures are required to force mercury into micropores. Higher pressures are required to measure the pore sizes and surface areas of the micropores present on the surfaces of silica products of the present invention. A suitable instrument for measuring micropore sizes and surface areas using mercury intrusion porosimetry for purposes of the present invention is a MICROMERITICS® Autopore II 9220 series automated mercury porosimeters, available from Micromeritics Instrument Corporation of Norcross, Ga.

The total quantity of cationic-compatible abrasive materials in the oral composition can be from about 1 to about 65%, preferably from about 3 to about 60%. In embodiments where the cationic-compatible abrasive is selected to be a dicalcium phosphate, the amount of abrasive present in the oral composition ranges from about 35 to about 60%. In embodiments where the cationic-compatible abrasive is selected to be a modified low surface area particulate, it is preferably present from between about 3 to about 25%.

In alternate embodiments, the cationic-compatible inorganic particulates having a porous surface substantially inert to the cationic active ingredients can be further pre-coated or encapsulated with an ethoxylated hydrogenated castor oil, such as those described in U.S. patent application U.S. Ser. No. 10/875,063 filed on Jun. 23, 2004, the contents of which are incorporated herein by reference.

Inorganic cationic-compatible particulate compounds which function as thickening agents may be used in the practice of the present invention, such as cationic-compatible colloidal silica inorganic particulate compounds. Further, the oral compositions of the present invention can include mixtures of cationic-compatible inorganic particulates, such as an abrasive and a colloidal thickener. Additionally, any suitable cationic-compatible inorganic particulates for safe use in an oral care composition are contemplated for use in the present invention.

Oral compositions of the present invention that include one or more cationic active ingredients are preferably stabilized in the aqueous solution by a surfactant system (i.e., a stabilizing surfactant system). By “stabilizing surfactant system”, it is meant that one or more surfactants are included in the oral composition that maintain or do not significantly decrease the bioavailability of the cationic active ingredient, including substantially limiting any potential hydrolysis or neutralization of the cationic portions of the active ingredient compound. Thus, in certain preferred embodiments of the present invention, the oral composition is aqueous and comprises a stabilized surfactant system having cationic-compatible surfactants selected from the group consisting of: non-ionic surfactants, cationic surfactants, zwitterionic surfactants, betaine surfactants, ampholytic surfactants, and mixtures thereof. In certain embodiments, mild and slightly anionic surfactants may be employed in the present invention, so long as there is not interference with the bioavailability of the cationic active ingredient, as will be discussed in greater detail below. Thus, one or more of such preferred classes of surfactants are included in the cationic-compatible surfactant system.

Nonionic surfactants are made of chemical constituents that result in a molecule having no ionic charges. As such, the nonionic surfactants are distinguished from cationic surfactants, anionic surfactants, and amphoteric surfactants. In a preferred embodiment, the hydrophilic moiety of a nonionic surfactant is based on a polyoxyalkylene structure. A polyoxyalkylene structure is a polyether type polymer that formally represents the polymerization product of a wide variety of cyclic ethers that polymerize by ring opening polymerization.

Non-ionic surfactants useful in the invention are usually synthesized by the polymerization of such cyclic ethers. Suitable nonionic surfactants useful in the present invention include poly(oxyethylene)-poly(oxypropylene) block copolymers. Such copolymers are known commercially by the non-proprietary name of poloxamers, the name is used in conjunction with a numeric suffix to designate the individual identification of each copolymer. Poloxamers may have varying contents of ethylene oxide and propylene oxide which results in poloxamers which have a wide range of chemical structures and molecular weights. A preferred poloxamer is Poloxamer 407, sold under the trade name PLURONIC® F127 by BASF, Inc. of Parsippany, N.J.

A preferred group of nonionic surfactants useful in the present invention include condensates of sorbitan esters of fatty acids with ethylene oxide (polysorbates) such as sorbitan mono-oleate with from about 20 to about 60 moles of ethylene oxide (e.g., TWEEN®, a trademark of ICI Group of Companies of London, England). Particularly preferred polysorbates are polyoxyethylene sorbitan monolaurate, such as Polysorbate 20 (polyoxyethylene 20 sorbitan monolaurate, TWEEN® 20) and Polysorbate 80 (polyoxyethylene 20 sorbitan monooleate, TWEEN® 80). In certain embodiments, Polysorbate 20 is particularly preferred.

Zwitterionic surfactants useful in the practice of the present invention, particularly betaines, include surfactants disclosed in U.S. Pat. No. 5,180,577 to Polefka, et al., the contents of which are incorporated herein by reference. Typical dimethyl glycine derivatives include alkyl diethyl betaines, such as, decal betaine 2-(N-decyl-N,N-dimethylammonio)acetate, cocobetaine or 2-(N-coc-N, N-dimethyl ammonio)acetate, myristyl betaine, palmityl betaine, lauryl betaine, cetyl betaine, cetyl betaine, stearyl betaine, and the like. The amido betaines are exemplified by cocoamidoethyl betaine, cocoamidopropyl betaine, lauramidopropyl betaine and the like. One preferred betaine is the cocoamidopropyl betaine.

In the present invention, non-ionic and zwitterionic surfactants are particularly preferred. However, anionic surfactants, where compatibilized with the cationic active ingredient compounds, may also be useful, particularly when they are mild and only slightly anionic surfactants. An example of one such preferred anionic surfactant is an acylated amino sulphonic acid, such as sodium methyl cocoyl taurate, also known as tauranol.

Thus, in various preferred embodiments, the cationic-compatible surfactant system comprises a surfactant selected from the group consisting of polyoxyethylene derivatives of sorbitan esters, dimethyl glycine derivatives, poly(oxyethylene)-poly(oxypropylene) block copolymers, acylated amino sulphonic acids, and mixtures thereof. More particularly, certain embodiments comprise a stabilizing surfactant system having a surfactant selected from the group consisting of: polyoxyethylene sorbitan monolaurate (polysorbate, for example, polysorbate 20), cocoamido propyl betaine (CAP betaine), poly(oxyethylene)-poly(oxypropylene) (poloxamer, for example, poloxamer 407), sodium methyl cocoyl taurate (tauranol), or mixtures thereof. In certain embodiments, a particularly efficacious stabilizing surfactant system comprises both a first surfactant of cocoamido propyl betaine (CAP betaine) and a second surfactant of (oxyethylene)-poly(oxypropylene)poloxamer, preferably poloxamer 407. In such embodiments, a preferred ratio of the first surfactant to the second surfactant ranges from about 1:0 to about 10:1 on a weight basis.

In various embodiments of the present invention, the surfactant(s) of the cationic-compatible stabilizing surfactant system are present in the oral composition in the range from of about 0.1% to about 5% by weight, preferably from about 0.6% to about 3% by weight.

In various embodiments of the present invention, the oral care composition has a delivery vehicle or carrier and further comprises additional ingredients. Preferably, the carrier does not reduce the efficacy of cationic active ingredients or other active materials of the present oral compositions. An acceptable vehicle or carrier in accordance with the present invention can be any carrier toxicologically suitable for use in the oral cavity. Such orally acceptable carriers include the usual components of toothpastes, tooth powders, prophylaxis pastes, mouth rinses, lozenges, gums and the like, and are more fully described hereinafter. Selection of specific carrier components is dependant on the desired product form, including dentifrices, rinses, gels, and paints. Preferred embodiments of the present invention include dentifrice oral compositions.

The pH of the oral composition carrier is preferably in the range of from about 4.5 to about 9. In particular embodiments, where the cationic active ingredient comprises an antibacterial ester, such as ELAH, it is preferred that the pH is acidic and is less than about 7.3, preferably less than about 6.8, more preferably less than about 6.5. The pH can be controlled by surfactant and ingredient selection, or by altering the carrier with acid (e.g., citric acid or benzoic acid) or base (e.g., sodium hydroxide) or buffered (with sodium citrate, benzoate, carbonate, or bicarbonate, disodium hydrogen phosphate, or sodium dihydrogen phosphate, for example).

In various embodiments, the carrier used to prepare the oral composition comprises water, preferably deionized water. The compositions of the present invention optionally include other materials such as adhesion agents, viscosity modifiers, diluents, foam modulators, pH modifying agents, humectants, cationic-compatible thickeners, mouth feel agents, sweeteners, flavorants, colorants, preservatives, and combinations thereof. It is understood that while general attributes of each of the above categories of materials may differ, there may be some common attributes and any given material may serve multiple purposes within two or more of such categories of materials. Preferably, such carrier materials are selected for compatibility with the cationic active material and other ingredients of the composition, as recognized by one of skill in the art. Conventional exemplary oral composition ingredients are described in U.S. Pat. No. 6,290,933 to Durga et al. and U.S. Pat. No. 6,685,921 to Lawlor, the contents of each of which are incorporated herein by reference.

For example, the compositions of the present invention preferably comprise a humectant, such as glycerin, sorbitol, xylitol, polyethylene glycol and/or propylene glycol of molecular weight in the range of 200 to 1,000. The humectant concentration typically totals about 5 to about 70% by weight of the oral composition. In certain preferred embodiments of the present invention, the oral composition comprises a humectant selected from glycerin and sorbitol that are present from about 8 to about 40%.

Thickeners used in the compositions of the present invention other than cationic-compatible silica thickeners discussed above, preferably include cationic-compatible natural and synthetic gums, colloids, and other inert materials such as polyethylene and crosslinked PVP. Suitable thickeners include naturally occurring polymers such as carrageenans, xanthan gum, synthetic thickeners such as polyglycols of varying molecular weights, cellulose polymers, such as hydroxyethyl cellulose and hydroxpropyl cellulose. The thickening agents are present in the oral compositions of the present invention in amounts of about 0.1 to about 10% by weight, preferably about 0.5 to about 4.0% by weight. In certain preferred embodiments, the thickeners used in the present invention, aside from cationic-compatible silica thickeners, include hydroxyethyl cellulose (HEC) that is optionally present from about 0 to about 5% and xanthan gum that is optionally present from about 0 to about 3%.

The oral composition of the present invention may also contain a flavoring agent, as are well known in the art. Suitable flavorants include essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Of these, the most commonly employed are the oils of peppermint and spearmint. The flavoring agent is incorporated in the dentifrice composition at a concentration of about 0.1 to about 5% by weight and preferably about 0.5 to about 2% by weight.

In certain embodiments of the present invention, multiple active ingredients in the oral composition are preferred, as they have been observed to improve the active ingredient (e.g., antibacterial) efficacy of the oral composition. The compositions of the present invention optionally comprise an additional active material, which is operable for the prevention or treatment of a condition or disorder of hard or soft-tissue of the oral cavity, the prevention or treatment of a physiological disorder or condition, or to provide a cosmetic benefit, the functions of which were previously described above in the context of the cationic active ingredients. These actives are either compatible with the cationic active ingredient(s) or are separately maintained from the cationic active ingredient(s) in a multiple component oral composition.

Oral care actives among those useful herein include whitening agents, antibacterial agents, antimicrobial agents, anti-inflammatory agents, anticaries agents, tartar control agents, antiplaque agents, periodontal actives, abrasives, breath freshening agents, malodour control agents, tooth desensitizers, salivary stimulants, and combinations thereof. It is understood that while general attributes of each of the above categories of actives may differ, there may some common attributes and any given material may serve multiple purposes within two or more of such categories of actives. If added in the same phase as the cationic active ingredient, the additional active ingredient should not react with or detract from the efficacy and bioavailability of the cationic active ingredient; preferably the additional ingredients are cationic or nonionic. Actives among those useful herein are disclosed in U.S. Patent Publication 2003/0206874 to Doyle et al., published Nov. 6, 2003; as well as in U.S. Pat. Nos. 6,290,933 and 6,685,921 to Lawlor, the contents of each of which are incorporated herein by reference.

In one embodiment, the oral compositions comprise additional non-cationic antibacterial agents. Such antibacterial agents include those based on phenolic and bisphenolic compounds, such as, halogenated diphenyl ethers, including triclosan (2,4,4′-trichloro-2′-hydroxy-diphenylether, triclocarban (3,4,4-trichlorocarbanilid), as well as 2-phenoxyethanol, benzoate esters, and carbanilides. Such additional antibacterial agents can be present in the oral care composition at quantities of from about 0.01 to about 5% by weight of the oral composition.

For example, in one embodiment where multiple active ingredients are included in the oral composition, an antibacterial ester such as ELAH is the cationic active ingredient, and an additional active ingredient is triclosan, where the oral composition is provided in a single or multiple component form.

The dentifrice composition of the present invention may also contain a source of fluoride ions or fluorine-providing component, as anticaries or antitartar agent in amount sufficient to supply about 25 ppm to 5,000 ppm of fluoride ions and include inorganic fluoride salts, such as soluble alkali metal salts. For example, preferred fluoride sources in the composition are sodium fluoride, potassium fluoride, sodium monofluorophosphate, sodium fluorosilicate, ammonium fluoro silicate, as well as tin fluorides, such as stannous fluoride. Sodium fluoride or sodium monofluorophosphate are preferred.

In addition to fluoride compounds, there may also be included stannous ion source antitartar agents such as stannous fluoride, stannous chloride, and stannous pyrophosphate.

In certain embodiments, antitartar agents comprise a zinc ion source such as zinc salts including zinc chloride, zinc citrate, zinc lactate, and zinc gluconate that are compatible with the cationic active ingredient. When antitartar agents are included in the oral composition, it is preferred that they are at a concentration of about 0.5% to about 5% by weight.

In certain embodiments of the present invention, the oral composition comprises a nonionic galenical extract in addition to the cationic active ingredient. Preferably such nonionic galenical extracts contain one or more active compounds derived from a natural source, preferably a plant source. The term galenical extract includes synthetic or semi-synthetic equivalents of such an extract or an active component contained therein. Extracts suitable for use in the present invention can be obtained from any part of the plant including the leaf, stem, bark, pulp, seed, flesh, juice, root and mixtures thereof. It is preferred that the extract is obtained from the leaf, pulp and seed, more preferably from the leaf, flower or bark.

Suitable non-limiting examples of nonionic galenical extracts include a hops acid extract from the hops plant or other part of a plant from the Cannabaceae family, in particular from the Humulus lupulus plant. Extracts of Magnolia Cortex (the bark of Magnolia officinalis) contain active compounds including: magnolol, honokiol, tetrahydromagnolol, and tetrahydrohonokiol, which are suitable for use in the present invention. Other antibacterial natural extracts include those isolated from green or oolong tea (Camellia sinensis), gold thread (derived from any of the following plant families: Annonaceae, Berberidaceae, Menispermaceae, Papaveraceae, Ranunculaceae, Rutaceae, Zingiberaceae, Nadina, Mahonia, Thalictrum spp.), cranberry and other Ericaceae family plants, honeysuckle (Lonicera ceprifolium), grape seed (Vitis vinifera), myrobalan (Terminalia bellerica), rosemary (Rosmarinus officinalis), East Indian walnut (Albizia lebbek), neem or margosa plant (Melia azadirachta), niruri (Phyllanthus niruri), and pine bark, preferably Maritime pine (Pinus pinaster). The Ericaceae family broadly refers to over 100 genera and the over 4,000 associated species, such as those disclosed in U.S. Pat. No. 5,980,869 to Sanker, et al. In certain embodiments, extracts from plants in the Vaccinium genus are useful as natural extracts, such as cranberry (Vaccinium macrocarpon).

Other natural extracts that may be antimicrobial and/or anti-inflammatory agents are those listed in the International Cosmetic Ingredient Dictionary and Handbook, Tenth Ed., 2004, page 2183. In various embodiments, the additional nonionic galenical extracts added to the oral composition of the present invention are preferably present from about 0.0001% to about 10%, preferably from about 0.001% to about 5%, more preferably from about 0.01% to about 3%, depending on the concentration of the cationic active compounds and form of the oral composition.

In various embodiments, the oral composition further comprises one or more additional active ingredients selected from the group: triclosan, stannous ion source, fluoride ion source, zinc ion source, nonionic galenical extracts, and mixtures thereof, in addition to the cationic active ingredients.

The oral composition of the present invention may be provided in a multi-component form having multiple phases that are maintained separately. For example, multiple component compositions are packaged in a suitable dispensing container in which the first and second components are maintained separately and from which the separated components may be dispensed synchronously as a combined ribbon for application to a toothbrush. Under such circumstances, it may be preferable that each component is formulated to have similar rheological characteristics, so that the two components may be simultaneously co-extruded in the desired predetermined amounts when separately housed in a multi-compartmented tube or pump device. Such containers are well known in the art. An example of such a container is a two compartment dispensing container, such as a pump or a tube, having collapsible sidewalls, as disclosed in U.S. Pat. No. 6,447,756 to Dixit, et al., U.S. Pat. No. 4,487,757 to Kiozpeoplou, and U.S. Pat. No. 4,687,663 to Schaeffer, where, the tube body is formed from a collapsible plastic web such as polyethylene or polypropylene and is provided with a partition within the container body defining separate compartments in which the physically separated components are stored and from which they are dispensed through a suitable dispensing outlet.

In the multiple component oral compositions of the present invention, the first component preferably comprises one or more cationic active ingredients. In various embodiments, the first component comprises a cationic-compatible inorganic particulate having a porous surface that is substantially inert to the cationic active ingredient, a cationic-compatible surfactant system, and a carrier that is stable in the presence of the cationic active ingredient, as previously described above.

The second component of the multiple component embodiment preferably contains one or more ingredients that are incompatible with the cationic active ingredient(s). For example, certain surfactants provide desirable foaming characteristics, however are too anionic to be stored with the cationic active ingredient. One example of such an anionic surfactant is sodium lauryl sulfate (SLS). Another example of such ingredients are certain antitartar agents that are generally recognized as not being compatible with cationic active ingredients, such as pyrophosphate and polyphosphate salts. Likewise, these ingreidents may be included in the second component. Other examples include conventional abrasives that are anionically charged.

Such conventional abrasives which may be used in the second component of the multi-component oral composition include conventional silica abrasives such as precipitated silicas having a mean particle size of up to about 20 microns, such as ZEODENT® 115, marketed by Huber Engineered Materials. Other useful anionic dentifrice abrasives include sodium metaphosphate, potassium metaphosphate, tricalcium phosphate, dihydrated dicalcium phosphate, aluminum silicate, calcined alumina, bentonite or other siliceous materials, or combinations thereof.

In other embodiments of the present invention, useful abrasive materials for preparing dentifrice compositions include silica gels and precipitated amorphous silica having an oil absorption value of less than 100 g/100 g silica and preferably in the range of from about 45 g/100 g to less than about 70 g/100 g silica. These silica colloidal particles have an average particle size ranging from about 3 microns to about 12 microns, and more preferably between about 5 to about 10 microns.

Further suitable abrasives useful with various embodiments of the present invention are low oil of absorption silica abrasives or high cleaning abrasives such as those marketed under the trade designation SYLODENT® XWA or SYLODENT® 783 by Davison Chemical Division of W. R. Grace & Co., Baltimore, Md. SYLODENT® 650 XWA, a silica hydrogel composed of particles of colloidal silica having a water content of 29% averaging from about 7 to about 10 microns in diameter, and an oil absorption of less than 70 g/100 g of silica is a preferred example of a low oil absorption silica abrasive useful in the practice of the present invention. Any other suitable oral care polishing materials can also be used in the second component of the present invention, as recognized by one of skill in the art. The abrasive is present in the second component of the dentifrice composition of the present invention at a concentration of about 10 to about 40%.

Thus, ingredients that are incompatible with the cationic active ingredient can be separately maintained in a second component of the oral composition. In a dual component oral composition system, the first component contains the cationic active ingredient and the second component contains the incompatible active ingredient. The first and second components are maintained separately from each other until dispersed for application to the oral cavity. Alternatively, such non-compatible active ingredient agents may also be included in a single phase dentifrice composition by compatibilization techniques recognized by one of skill in the art, such as providing a low concentration of water to physically separate and prevent diffusion of the non-compatible ingredients thus diminishing contact between them.

Likewise, certain active ingredients may have improved bioavailability or efficacy when delivered stored or delivered with ingredients that potentially destabilize the cationic active ingredients. One such example is triclosan, which is highly efficacious when delivered with an anionic polycarboxylate and sodium lauryl sulfate. However, the SLS and anionic polycarboxylate are incompatible with the cationic active ingredients.

Synthetic anionic polycarboxylates are often used in dentifrice compositions as an efficacy enhancing agent for certain active ingredients, including antibacterial, antitartar or other active agents within the oral composition. Such anionic polycarboxylates are generally employed in the form of their free acids or preferably partially or more preferably fully neutralized water soluble alkali metal (e.g., potassium and preferably sodium) or ammonium salts. Preferred copolymers are are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. One preferable copolymer is methylvinylether/maleic anhydride. Examples of these copolymers are available from ISP Corporation under the tradename GANTREZ®, e.g., AN 139 (M.W. 1,100,000), AN 119 (M.W. 200,000); S-97 Pharmaceutical Grade (M.W. 1,500,000), AN 169 (M.W. 2,000,000), and AN 179 (M.W. 2,400,000); wherein the preferred copolymer is S-97 Pharmaceutical Grade (M.W. 1,500,000).

In certain embodiments, a multi-component oral care composition for application to an oral surface comprises a first phase or component having one or more cationic active ingredients, where at least one of the cationic ingredients is ethyl lauroyl arginine ester (ELA). The first phase also comprises a cationic-compatible inorganic particulate having a porous surface that is substantially inert to the cationic active ingredient, a cationic-compatible surfactant system, and a carrier having a pH of less than about 7. The second phase or component comprises one or more oral care ingredients incompatible with the cationic antibacterial ingredient. In certain embodiments, the second phase comprises one or more of: an anionic surfactant, an anionic active ingredient, and a particulate incompatible with the cationic active ingredient. The first and second phases are maintained separately from one another during storage. In one particular embodiment, the second phase comprises one or more of: sodium lauryl sulfate and a particulate incompatible with the cationic active ingredient. In another embodiment, the second phase comprises triclosan and a copolymer of methyl vinyl ether and maleic anhydride. The second phase may further comprise a particulate incompatible with the cationic active ingredient.

In one embodiment, the present invention provides a method of making a dentifrice having an enhanced availability and stability of a cationic oral care active ingredient. The preparation of dentifrices is well known in the art.

More specifically, one exemplary method of preparing a dentifrice of the present invention generally includes dispersing water soluble compounds in water, including components such as, sodium saccharin, monofluorophosphate, and any other salts, that are mixed in a conventional mixer under agitation. The humectants e.g., glycerin and sorbitol, are dispersed in water in a conventional mixer under agitation. The organic thickeners, such as xanthan gum and hydroxyethylcellulose, and any polymers, are added. The resultant mixture is agitated until a homogeneous gel phase is formed. Into the gel phase are added a pigment such as TiO₂, and any acid or base required to adjust the pH to 6 to 7. The mixture is then transferred to a high-speed vacuum mixer, where the cationic-compatible inorganic particulate abrasives and/or thickeners, such as modified low-surface area silica or dicalcium phosphate, are added. The mixture is then mixed at high speed for from 5 to 30 minutes, under vacuum of from about 20 to 50 mm of Hg, preferably about 30 mm Hg. The flavor oil is weighed out and then added to the mixture. Lastly, the surfactants of the stabilizing surfactant system (sodium methyl cocoyl taurate, i.e., tauranol, TWEEN® 20, cocoamido betaine, i.e., CAP betaine, PLURONIC®127, i.e., poloxamer 407), flavor, and cationic active ingredient, (i.e., ELAH) and any other active ingredients are added to the mixture and mixed for an additional 10 minutes. The resultant product is a homogeneous, semi-solid, extrudable paste of gel product.

The following examples further describe and demonstrate preferred embodiments within the scope of the present invention.

### EXAMPLE 1

TABLE 1

| | Formula | Formula | Formula |
|---|---|---|---|
| Ingredient | A | B | C |
| Cationic-compatible silica | 15 | 18 | |
| thickener | | | |
| Dicalcium Phosphate | | | 48.8 |
| dehydrate | | | |
| Glycerin | 11 | 11 | 18 |
| Sorbitol | 20 | 20 | 15.1 |
| Hydroxyethylcellulose | 1.3 | 1.3 | 1 |
| Xanthan gum | | | |
| MFP | 0.76 | 0.76 | 0.76 |
| Sodium saccharin | 0.3 | 0.3 | 0.3 |
| NaOH (50%) | qs | qs | |
| Flavor | 1.2 | 1.2 | 0.7 |
| Ethyl Lauroyl Arginate | 0.8 | 0.8 | 0.8 |
| Hydrochloride (ELAH) | | | |
| (20%) | | | |
| Sodium Methyl Cocoyl | | | |
| Taurate (95%) | | | |
| TWEEN ® 20 | | | |

Each dentifrice formulation in Table 1 is prepared by dispersing water soluble salts and compounds, sodium saccharin sodium and monofluorophosphate (MFP) in a conventional mixer under agitation. The humectants e.g., glycerin and sorbitol, are dispersed in water in a conventional mixer under agitation. The organic thickeners, such as xanthan gum and hydroxyethylcellulose, are added. The mixture is agitated until a homogeneous gel phase is formed. Into the gel phase is added titanium dioxide pigment, such as TiO₂, and any acid or base (NaOH) required to adjust the pH to less than about 7. The mixture is then transferred to a high-speed vacuum mixer, where the cationic-compatible inorganic particulate abrasives and/or thickeners, such as modified low-surface area silica or dicalcium phosphate, are added. The mixture is then mixed at high speed for from 5 to 30 minutes, under vacuum of from about 20 to 50 mm of Hg, preferably about 30 mm Hg. The flavor oil is weighed out and then added to the mixture. Lastly, the surfactants of the stabilizing surfactant system (sodium methyl cocoyl taurate, i.e., tauranol, TWEEN® 20, cocoanido betaine, i.e., CAP betaine, PLURONIC®127, i.e., poloxamer 407), flavor, and cationic active ingredient, (i.e., ELAH) is added to the mixture and mixed for an additional 10 minutes. The resultant product is a homogeneous, semi-solid, extrudable paste or gel product.

### EXAMPLE 2

TABLE 2

Formula
Dual Tube

| Ingredients | Side 1 | Side 2 |
|---|---|---|
| Cationic-compatible silica | | |
| abrasive (Grace) | | |
| Cationic-compatible silica | | |
| thickener (Grace) | | |
| ZEODENT ® 115 | 10 | |
| ZEODENT ® 165 | 3.5 | |
| XWA 650 | 10 | |
| Dicalcium Phosphate | | 48.8 |
| Dihydrate DF | | |
| Glycerin | 20 | 18 |
| Sorbitol | 16.9 | 15.1 |
| Hydroxyethylcellulose | | 1 |
| Gum | | |
| Propylene Glycol | 0.5 | |
| CMC Gum | 1.1 | |
| (CarboxyMethyl | | |
| Cellulose) | | |
| Iota Carageenan Gum | 0.5 | |
| NaF | 0.243 | 0.243 |
| Sodium saccharin | 0.3 | 0.3 |
| NaOH (50%) | 1.2 | |
| Flavor | 1 | 1 |
| Solbrol (20%) | | 7.5 |
| Triclosan | 0.6 | |
| GANTREZ ® | 4 | |
| Sodium Lauryl Sulfate | 3 | |
| TWEEN ® 20 | | 1 |
| CAP Betaine | | 1 |
| PLURONIC ® 127 | | |
| (Poloxamer 407) | | |
| Titanium dioxide | 0.5 | 0.5 |
| Water | Balance | Balance |

Multi-component dentifrice compositions are prepared as shown in Table 2. The first component or phase was prepared as described in Example 1. The second component is prepared by adding the water soluble compounds, such as MFP and sodium saccharin to water and mixing. It should be noted that fluoride ion sources can be added to both the first and second components. Then the humectants are added and mixed under agitation. Into the dispersion is added the thickeners, such as xanthan gum and/or hydroxyethyl cellulose. Then GANTREZ® is added with sodium hydroxide. The resultant mixture is agitated until a homogeneous gel phase was formed. Into the gel phase are added titanium dioxide (TiO₂) and buffering agents. These ingredients are mixed until a homogenous phase is obtained. The mixture is then transferred to a high speed/vacuum mixer; wherein the triclosan, GANTREZ®, and conventional abrasive compounds, such as ZEODENT®115, ZEODENT®165, high cleaning silica, SYLODENT® XWA 650, are added and mixed at high speed for 25 minutes, under vacuum from about 30 mm Hg. Finally, the surfactant(s), including the anionic surfactant, sodium lauryl sulfate (SLS), and then flavor are added to the mixture and mixed for an additional 10 minutes. The resultant product is a homogeneous, semisolid, extrudable paste or gel product. The first and second components are stored separately in a multi-component storage device, such as those described previously above.

What is claimed is:

1. An oral composition comprising:
(a) a cationic active ingredient;
(b) a cationic-compatible inorganic particulate having a surface that is substantially inert to the cationic active ingredient; and
(c) a cationic-compatible surfactant system.

2. An oral composition according to claim 1, wherein the cationic-compatible inorganic particulate component comprises a silica particulate that has a BET surface area of less than about 100 m²/g.

3. An oral composition according to claim 1, wherein the cationic-compatible inorganic particulate component comprises a DBP oil of absorption of less than about 150 g/100 g.

4. An oral composition according to claim 1, wherein the cationic-compatible inorganic particulate component comprises a modified low-surface area silica particulate that has a median diameter of less than about 100 micrometers and the BET surface area is less than about 50 m²/g.

5. An oral composition according to claim 1, wherein the cationic-compatible inorganic particulate comprises a dicalcium phosphate particulate.

6. An oral composition according to claim 1, wherein the cationic active ingredient is selected from benzethonium chloride, octenidine, hexetidine, hexamidine, chlorhexidine, alexidine, and N′-acyl arginine alkyl ester salts.

7. An oral composition according to claim 1, wherein the cationic active ingredient comprises cetyl pyridinium chloride.

8. An oral composition according to claim 5, wherein the cationic active ingredient comprises an ethyl lauroyl arginine ester hydrochloride (ELAH).

9. An oral composition according to claim 1, wherein the oral composition further comprises an additional active ingredient selected from the group consisting of: triclosan, stannous ion source, fluoride ion source, zinc ion source, and nonionic galenical extracts.

10. An oral composition according to claim 1, wherein the cationic active ingredient is present at a concentration of between about 0.05 to about 5% by weight of the oral care composition.

11. An oral composition according to claim 1, wherein the cationic-compatible surfactant system comprises a surfactant selected from the group consisting of: non-ionic surfactants, cationic surfactants, betaine surfactants, and ampholytic surfactants.

12. An oral composition according to claim 1, wherein the cationic-compatible surfactant system comprises a surfactant selected from the group consisting of: polyoxyethylene sorbitan monolaurate, cocoamido propyl betaine, poly(oxyethylene)-poly(oxypropylene)(poloxamer), sorbitan diisostearate, alkyl polyglucoside, and sodium methyl-cocoyl taurate.

13. An oral composition according to claim 1, wherein the cationic-compatible surfactant system comprises a first surfactant of poly(oxyethylene)-poly(oxypropylene)(poloxamer) 407 and a second surfactant of cocoamido propyl betaine, wherein a ratio of the first surfactant to the second surfactant ranges from about 1:0 to about 10:1 on a weight basis.

14. An oral composition according to claim 1, further comprising a dentifrice ingredient selected from the group consisting of: humectants, surfactants, cationic-compatible thickening agents, water, flavoring agents, and preservatives.

15. An oral composition comprising:
(a) a compound represented by formula (I) 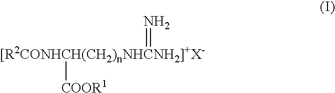
where R¹ and R² are alkyl groups, and X is an anion;
(b) a cationic-compatible inorganic particulate having a porous surface that is substantially inert to the cationic antibacterial ester; and
(c) a cationic-compatible surfactant system comprising a surfactant selected from the group consisting of: polyoxyethylene sorbitan monolaurate, cocoamido propyl betaine, poly(oxyethylene)-poly(oxypropylene)(poloxamer), and sodium methyl cocoyl taurates.

16. An oral composition according to claim 15, wherein R¹ is an alkyl chain of 1 to 8 carbon atoms and R² is an alkyl chain of 6 to 30 atoms.

17. An oral composition according to claim 15, wherein the cationic-compatible inorganic particulate component comprises a low surface area silica particulate that has a BET surface area of less than about 100 m²/g.

18. An oral composition according to claim 15, wherein the cationic-compatible inorganic particulate component comprises a DBP oil of absorption of less than about 150 g/100 g.

19. An oral composition according to claim 15, wherein the cationic-compatible inorganic particulate component comprises a modified low-surface area silica particulate that has large diameter pores with a diameter of greater than 500 Angstrom, wherein the large diameter pores have a cumulative surface area as measured by mercury intrusion of less than about 8 m²/g.

20. An oral composition according to claim 15, wherein the cationic-compatible inorganic particulate comprises a dicalcium phosphate particulate.

21. An oral composition according to claim 15, wherein the cationic antibacterial ester comprises an ethyl lauroyl arginine ester hydrochloride (ELAH).

22. An oral composition according to claim 15, wherein the oral composition further comprises an additional active ingredient selected from cetyl pyridinium chloride, chlorhexidine, stannous ion source, fluoride ion source, zinc ion source, and galenical extracts.

23. An oral composition according to claim 15, wherein the cationic-compatible surfactant system comprises a surfactant selected from the group consisting of: polyoxyethylene sorbitan monolaurate, cocoamido propyl betaine, poly(oxyethylene)-poly(oxypropylene)(poloxamer), and sodium methyl cocoyl taurate.

24. An oral composition according to claim 15 further comprising triclosan.

25. An oral care composition for application to an oral surface, the composition comprising:
(a) a first phase comprising a cationic active ingredient, a cationic-compatible inorganic particulate having a surface that is substantially inert to the cationic active ingredient, and a cationic-compatible surfactant system; and
(b) a second phase comprising one or more oral care ingredients incompatible with the cationic antibacterial ingredient, wherein the second phase comprises one or more of: an anionic surfactant, an anionic active ingredient, and a particulate incompatible with the cationic active ingredient, wherein the first and the second phases are separated from one another during storage.

26. An oral care composition according to claim 25, wherein the second phase comprises one or more of: sodium lauryl sulfate and a particulate incompatible with the cationic active ingredient.

27. An oral care composition according to claim 25, wherein the second phase comprises triclosan and a copolymer of methyl vinyl ether and maleic anhydride.

28. An oral care composition according to claim 25, wherein the cationic-compatible inorganic particulate component comprises a silica particulate that has a BET surface area of less than about 100 m²/g.

29. An oral care composition according to claim 25, wherein the cationic-compatible inorganic particulate component comprises a DBP oil of absorption of less than about 150 g/100 g.
